(19) United States
(12) Patent Application Publication
(10) Pub. No.: US 2011/0111060 A1
(43) Pub. Date: May 12, 2011

(54) COMPOSITIONS, FORMULATIONS, AND METHODS FOR PREVENTING AND/OR TREATING PHYSICAL EFFECTS OF ALCOHOL CONSUMPTION

(76) Inventor: Andrew R. Chadeayne, Arlington, VA (US)

(21) Appl. No.: 13/001,485

(22) PCT Filed: Jun. 24, 2009
(86) PCT No.: PCT/US09/48415

§ 371 Date: Dec. 27, 2010

Related U.S. Application Data
(60) Provisional application No. 61/076,009, filed on Jun. 26, 2008.

(51) Int. Cl. A61K 33/14; C01D 3/04; A61P 43/00

(52) U.S. Cl. 424/680; 423/499.4

(57) ABSTRACT

The present invention provides effective novel compositions and formulations for treating and/or preventing a hangover. The present invention also provides methods for treating and/or preventing a hangover comprising administering the compositions and/or formulations of the present invention.

### TECHNICAL FIELD

The invention relates generally to the prevention and/or treatment of physical effects associated with the consumption of alcohol.

### BACKGROUND

In some people, the consumption of alcoholic beverages may cause a condition known as an “alcohol hangover,” or simply a “hangover.” The term “hangover” refers to an array of symptoms that affect a person shortly after consuming alcoholic beverages. The symptoms that define a hangover have been the subject of some dispute in the scientific community, but may include, for example, a combination of one or more of the following symptoms: thirst, fatigue, headache, dizziness/faintness, loss of appetite, stomach ache, nausea, and elevated heart rate. See Rohsenow, D. J. et al., Addictive Behaviors, 2007, 32: 1314-20.

Alcoholic beverages are drinks that contain the molecule ethanol, which is represented by the chemical formula C₂H₅OH. Ethanol is the primary psychoactive ingredient in alcoholic beverages including wine, beer, and distilled spirits. An estimated 70 million Americans consume alcoholic beverages. Dalessio, D. J., 1973, Headache: The Journal of Head and Face Pain, 12(4): 173-176. Owing to the popularity of alcoholic beverages, ethanol is probably one of the most popular drugs in the world. A hangover is considered the most widely experienced negative consequence of consuming ethanol. See Wechsler et al., JAMA, 1994, 272: 1672-77. A hangover, which may occur following non-problem or “social drinking,” is distinct from withdrawal symptoms that accompany so-called “problem drinking.” See Dalessio, D. J. at 174. Because the hangover results in symptoms such as headache and nausea, it often reduces a person's ability to perform tasks effectively throughout the duration of the hangover. For example, professionals may experience a hangover at work the morning following a nighttime social engagement during which alcohol was consumed. Accordingly, hangovers result in substantial economic costs due to losses in productivity within the socially-drinking workforce. See Stockwell, Alcohol Clin. Exp. Res., 2nd Sup., 1998, 22: 63S-69S; Crofton, Alcohol, 1987, 22: 321-25. Despite the discomfort experienced by the person afflicted with a hangover and the cost of the hangover to society, relatively little research has been done to reliably reduce the negative effects of a hangover. See Swift and Davidson, Alcohol Health Res. World, 1998, 22: 54-60.

While the scientific community now agrees on most of the symptoms that describe a hangover, very little progress has been made with respect to alleviating these symptoms. Id. This lack of progress is due, in part, to a poor understanding of the causes that underlie the hangover. Peterson, J. V. et al, A Nation Under the Influence, 2003, A and B: New York, 22. Many conflicting theories attribute the entire array of hangover symptoms to a single underlying cause. Some of the more popular hypotheses for the causes of a hangover include dehydration, electrolyte imbalance, gastrointestinal disturbances, hypoglycemia, sleep disruption and/or deprivation, congeners (i.e., impurities in the alcoholic beverage), and metabolites of ethanol (e.g., acetaldehyde). As several of these theories are believed to be at least partially accurate, they often receive recognition in scientific journals as allowing a particular solution to the problem. Unfortunately, each individual contribution is often overemphasized and touted as a panacea, or hangover cure, which discredits earlier, seemingly conflicting work in the area. As a result of the rise and fall of disparate theories for the cause of a hangover, efforts to develop an effective composition for preventing and/or treating the hangover have been frustrated. Accordingly, effective methods for preventing and/or treating the hangover remain elusive.

As a result of the relative lack of scientific scrutiny and the potential for multiple causes underlying a hangover, an effective treatment and/or prevention for the most egregious manifestations of a hangover, generally considered to be headache and/or nausea, has gone unrealized. Despite the relative ineffectiveness of most marketed hangover remedies, the market for potential remedies continues to expand, indicating that there is a significant unmet need for an effective means for treating and/or preventing a hangover. The following website, called “The Hangover Review” summarizes and reviews hangover remedies on the present market: www.hangoverreview.com. This website hypothesizes that the increasing number of proffered hangover cures is the result of “me too” marketing, playing into society's desire to obtain an effective remedy and willingness to try anything held out as a promising cure. Many products entering the market are merely a blend of old, ineffective remedies and/or are made of a scientifically unsupported “snake oil” ingredient. For example, the leading ingredient in some presently marketed hangover remedies is vitamin C, which is present in 16 different products. In addition, a recent formulation contends that “Wild Guava Leaf extract is the story and secret that makes Drinkin' Mate effective.” See www.drinkinmate.com/focusgroups.php.

To date, however, there is no effective method for reliably preventing and/or alleviating the hangover, which arises following the consumption of one or more alcoholic beverages. Particularly, despite the large number of products on the market and variety of ingredients, no hangover prevention and/or treatment product is effective in mitigating the symptoms of a hangover, such as, for example, headache and nausea/vomiting. Providing a reliable composition, formulation and/or method of treating and/or preventing a hangover would allow social drinkers to enjoy consuming alcoholic beverages without incurring such negative physical aftereffects and potential loss in productivity.

The present invention provides effective novel compositions and formulations for treating and/or preventing a hangover. The present invention also provides methods for treating and/or preventing a hangover comprising administering the compositions and/or formulations of the present invention.

Without wishing to be bound by any theory, it is hypothesized that certain elements of an alcohol hangover, for example headache and nausea, may be due to an electrolyte imbalance, particularly an imbalance of sodium, potassium, and/or chloride ions in the body's circulating fluids. On average, a human (also referred to herein as a person) comprises about 40 L of circulating fluid volume. Within this fluid volume, the human body typically maintains a relatively narrow range of electrolyte concentrations. For example, a human typically maintains a potassium concentration within the range of approximately 3.5-5 mM; a sodium concentration within the range of approximately 135-145 mM; and a chloride concentration within the range of approximately 98-108 mM. When a person drinks or otherwise consumes alcoholic beverages, the ethanol in these beverages causes that person to urinate more frequently, resulting in the loss of both water and electrolytes. However, by their nature, beverages are liquids, comprising water, which provides at least some compensatory (re)hydration. However, most alcoholic beverages do not contain enough electrolytes to compensate for the body's loss of electrolytes during a period of drinking. For example, a can of Bud Light® beer contains 355 mL of water but only 9 mg of sodium. Accordingly, when a person drinks several cans of light beer, resulting in increased urination, s/he loses both water and electrolytes, yet replenishes essentially only the lost water. As a result, without proper supplementing, a person develops an electrolyte imbalance during drinking. The severity of the electrolyte imbalance increases as a function of the amount of beverage consumed.

The hangover cures presently on the market do not adequately address the severe electrolyte imbalance caused by consuming multiple alcoholic beverages. For example, Chaser®, one leading hangover remedy on the market, consists of activated calcium carbonate and vegetable carbon. See, e.g., U.S. Pat. No. 6,827,932. These ingredients aim to prevent the adverse effects of acetaldehyde, the first metabolite in the breakdown of ethanol. However, these ingredients do not address the issue of electrolyte imbalance.

Sobrietol®, another leading hangover remedy, allegedly reduces the severity of hangovers by enzymatically increasing the metabolism of ethanol and acetaldehyde in the blood. See U.S. Pat. No. 5,759,539. Like activated carbon, the enzymes intended to speed up the metabolism of ethanol and its metabolites do not effectively correct any electrolyte imbalance incurred by drinking. Other popular remedies and their ingredients are summarized at www.hangoverreview.com.

As discussed above, the hangover remedies currently available do not provide enough electrolytes to replenish those lost upon consumption of alcohol, particularly during an episode of moderate to heavy alcohol consumption. Moreover, beverages marketed for replenishing electrolytes lost under other circumstances such as through physical exercise may also not provide enough or the right balance of electrolytes to sufficiently allay an impending hangover. For example, Gatorade® provides 110 mg of sodium and 30 mg of potassium per serving. However, in one example a person with approximately 40 L of circulating volume dilutes his electrolyte concentration by up to about 1 g of sodium per 12 oz light beer. Accordingly, in this exemplary case, a person drinking light beer would need to consume approximately 9 servings of Gatorade® per serving of light beer consumed to effectively replenish lost electrolytes. As each serving of Gatorade has about 250 mL of water, this would require a person drinking light beer to consume an amount of Gatorade with 2.25 liters of water to replenish the electrolyte dilution incurred after approximately 1 light beer. The amount of water accompanying the electrolytes would further compound the electrolyte deficiency, making the problem worse. Fortunately, most people reabsorb electrolytes from the body fluid before they pass into the urine. Accordingly, the typical person's electrolyte depletion is not as severe as the most extreme case. Nonetheless, the principle is the same: consuming alcoholic beverages, such as beer, leads to electrolyte depletion, which is not adequately corrected by standard electrolyte solutions. Also, many people have trouble ingesting a large volume of fluid after an extended period of drinking because they feel “full.”

Other beverages specifically aimed to replenish electrolytes also suffer from an electrolyte concentration limitation. For example, Pedialyte®, Lytren®, and/or Rehydrate® are formulated to replace both the fluids and electrolytes lost during illness, such as through vomiting and diarrhea. Accordingly, the concentration of these beverages is higher in electrolytes and lower in sugar than sports drinks. Nonetheless, these formulations are still minimally effective in preventing and/or treating a hangover because the user must consume additional water in order to ingest the desired electrolytes. Additionally, in some cases, the lower sugar content prevents the formulation from alleviating the hypoglycemia induced by some instances of alcohol consumption. As a person's electrolyte concentration is low after drinking because of consuming too much water and not enough electrolytes, requiring a person to consume additional water in order to replenish electrolytes may be counterproductive from both a concentration and ease-of-use perspective.

The compositions and formulations of the present invention, however, provide an effective means for efficiently replenishing a person's electrolyte concentration, without requiring the ingestion of large volumes of water.

The methods of treating and/or preventing hangovers of the present invention are distinct from commonly practiced methods because they address the need for restoring the body's electrolyte concentration by administering appropriate doses of electrolytes. By administering the appropriate doses of electrolytes, the methods of the invention may prevent and/or treat a person's depleted electrolyte concentration, which may thereby mitigate the adverse effects (e.g., hangover and/or nausea) of, for example, hyponatremia.

Each of the salts and sugars useful in the invention may be readily attained from a chemical supplier such as Sigma-Aldrich. Additionally, many of the salts and/or sugars of the invention are readily available in supermarkets, health food stores, and pharmacies.

The term “alcohol” as used herein means the specific alcohol ethanol. While “alcohol” and “ethanol” may be used interchangeably to indicate the molecule of formula C₂H₅OH, neither are intended to be limited to the pure form of ethanol. To the contrary, any reference to alcohol and/or ethanol should be construed to include compositions (e.g., alcoholic beverages) comprising alcohol, such as beer, wine, liquor, etc., which are typically consumed by a human.

The term “salt” as used herein is intended to include the ion pair form of a salt (e.g., sodium citrate, NaCl, sodium glutamate, NaHCO₃, Na₂CO₃, K₂CO₃, KHCO₃, CaCO₃, KCl, etc.) and/or the solvated (e.g., NaCl_{aq}) or ion (e.g., Na⁺ or Cl⁻) forms. Where a quantity of salt is specified by its mass, the mass refers to the recited components. For example, 1 g of “sodium” refers to 1 g of Na⁺ as calculated by the mass of sodium whereas 1 g of “sodium chloride” refers to 1 g of NaCl, calculated by the mass of sodium chloride. Where a salt of a polyprotic acid (e.g., phosphoric acid) is specified (e.g., sodium phosphate), it should be understood that the salt specified embraces other degrees of deprotonation. For example, the term “sodium phosphate” should be construed to include mono-, di-, and tri-basic sodium phosphate. One of ordinary skill in the art will understand that these forms will exist in equilibrium with one another and their exact ratios will depend on, for example, the pH of the solution.

By way of example only, salts that may be used in the compositions, formulations, and methods of the invention may include a combination of one or more cation(s) with one or more anion(s), wherein the one or more cations may be chosen from, for example, Na⁺, K⁺, Ca²⁺, Zn²⁺, or Mg²⁺; and the one or more anions may be chosen from, for example, Cl⁻, HCO₃⁻, I⁻, PO₄³⁻, HPO₄²⁻, H₂PO⁴⁻, CO₃²⁻, HCO₃⁻, CH₃COOO⁻. For example, in the case of sodium, Na⁺, some exemplary salts that may be used within the context of the invention include Sodium Acetate, Sodium Acid Tartate, Sodium Acid Tartrate, Sodium Adipate, Sodium Alginate, Sodium Benzoate, Sodium Bicarbonate, Sodium Bromate, Sodium Carbonate, Sodium Chloride, Sodium Citrate, Sodium Dihydrogen Citrate, Sodium Dihydrogen Phosphate, Sodium Ferrocyanide, Sodium Gibberellate, Sodium Gluconate, Sodium Glycerophosphate, Sodium Hydroxide, Sodium Iodate, Sodium Iodide, Sodium Lactate Sodium Metabisulfite, Sodium Nitrate, Sodium Nitrite, Sodium Persulfate, Sodium Phosphate (Dibasic), Sodium Phosphate (Monobasic) Sodium, Phosphate (Tribasic), Sodium Polymetaphosphate, Sodium Pyrophosphate, Sodium Saccharin, Sodium L(+)-Tartrate, Sodium Sorbate, Sodium Sulfate, Sodium Sulfite, Sodium Tripolyphosphate. One of skill in the art will appreciate, however, that other cations and anions will be useful in the compositions, formulations, and methods of the invention.

The term “hangover” as used herein is intended to mean generally negative or unpleasant physiological symptoms that may arise in a person as a result of and following consumption of ethanol. By way of example only, the term “hangover” within the context of this invention may refer to the headache and nausea which often follow a period of elevated (e.g., greater than about 0.01%) blood alcohol concentration (“BAC”). Such elevated BACs generally occur following drinking or otherwise consuming alcohol. The onset of the hangover may depend on the peak BAC during the period of drinking, and may appear as the BAC returns to zero, for example lasting several hours thereafter. See Swift and Davidson 1998; Wiese et al., 2000, Annals of Internal Medicine, 132: 897-902. The term “hangover” is not intended to embrace alcohol withdrawal which arises from chronic (as opposed to acute) alcohol use.

The term “blood alcohol concentration” (or “BAC”) as used herein is intended to mean the mass of alcohol in the blood per unit of blood volume. For example, a BAC of 0.02% means 0.02 g of ethanol per 1 L of blood. A person's BAC can be easily determined by various methods, such as, for example, directly from analysis of the person's blood or by using a breathalyzer device, which may be readily attained in pharmacies and/or internet shopping sites.

The term “sugar” as used herein is intended to refer to any monosaccharide or disaccharide. The term “sugar” is not intended to be limited to its non-scientific meaning of only sucrose. The term “sugar” may also be used to describe mixtures of two or more sugars in any ratio, such as, for example, a mixture of sucrose and glucose.

The term “composition” as used herein refers to an admixture of one or more ingredients chosen from a sodium salt, a citrate salt, a phosphate salt (e.g., mono-, di-, tri-basic phosphate salts or mixtures thereof), a glutamate salt, a bicarbonate salt, a chloride salt, a carbonate salt, activated carbon, a sugar, a calcium salt, a potassium salt, and glycerol, optionally further comprising at least one suitable additive. In various exemplary embodiments, the composition may be appropriate for filling a capsule, making a tablet, or preparing some other type of formulation comprising the composition. Compositions according to the invention may be made in any way known to those of skill in the art.

The term “additive” as used herein refers to any inert or relatively inert component used for preparing compositions, such as, by way of example only, preservatives, binders, stabilizers, coloring agents, and flavoring agents.

The term “formulation” as used herein refers to compositions described herein that are further processed or formulated into a dosage form for consumption by a person. By way of example only, in various exemplary embodiments, the formulations may comprise a solid dosage form such as a capsule or tablet. In further exemplary embodiments, the formulations may comprise a liquid dosage form such as a gel, solution, concentrate, or beverage-like formulation. Formulations according to the invention may be made in any way known to those of skill in the art.

The term “powder” as used herein refers to all particulate solid forms of a material, including crystalline and amorphous forms.

The term “tablet” as used herein refers to a solid composition that is compressed or otherwise formed into a defined shape and quantity. For example, a powder may be compressed into a tablet by using excipients or binders to cause the powder to adhere to itself upon compressing. A tablet may be consumed directly, or used by adding the tablet to a liquid, for example dissolving or dispersing the composition throughout the liquid.

The term “pill” as used herein is intended to include a tablet, capsule, or other similar solid formulation that is intended, for example, to be swallowed directly upon administration. The term “pill” may also include, for example, a formulation in a pill-like form that is intended to be placed in the mouth and allowed to melt or put into a beverage and allowed to dissolve, such as a lozenge.

The terms “treat” and “prevent” (and variants thereof, such as “treatment” and “prevention”) as used herein are intended to be interchangeable and to encompass treating, alleviating, and/or preventing, to any degree, the symptoms and conditions associated therewith.

The terms “drink” or “drinking” as used herein when referring to the consumption of alcohol are intended to include the intake of alcohol in any form, including by drinking or otherwise ingesting or consuming alcohol.

In the following description, certain aspects and embodiments of the invention will become evident. It should be understood that the invention, in its broadest sense, could be practiced without having one or more features of these aspects and embodiments. It should be understood that these aspects and embodiments are merely exemplary and explanatory, and are not restrictive of the invention as claimed.

### DESCRIPTION OF EXEMPLARY EMBODIMENTS

Reference will now be made in greater detail to various exemplary embodiments of the invention, which are illustrated in the following description and examples. In various embodiments of the invention, hangover prevention and/or treatment compositions and/or formulations comprising a sodium salt, and optionally comprising at least one additional component chosen from a citrate salt, a phosphate salt (e.g., mono-, di-, tri-basic phosphate salts or mixtures thereof), a glutamate salt, a bicarbonate salt, a chloride salt, a carbonate salt, activated carbon, a sugar, a calcium salt, a potassium salt, and glycerol, are disclosed. In further exemplary embodiments of the invention, methods for preparing hangover prevention and/or treatment compositions and/or formulations are disclosed. In further exemplary embodiments of the invention, methods for preventing and/or treating a hangover comprising administering compositions and/or formulations comprising a sodium salt, and optionally comprising at least one additional component chosen from a citrate salt, a phosphate salt (e.g., mono-, di-, tri-basic phosphate salts or mixtures thereof), a glutamate salt, a bicarbonate salt, a chloride salt, a carbonate salt, activated carbon, a sugar, a calcium salt, a potassium salt, and glycerol, are also disclosed.

For example, a hangover prevention and/or treatment composition comprising a sodium salt is disclosed.

In another exemplary embodiment, a hangover prevention and/or treatment composition comprising a sodium salt and additionally comprising a sugar is disclosed.

In another exemplary embodiment, a hangover prevention and/or treatment composition comprising a sodium salt and additionally comprising a bicarbonate salt is disclosed.

In another exemplary embodiment, a hangover prevention and/or treatment composition comprising a sodium salt and additionally comprising a citrate salt is disclosed.

In another exemplary embodiment, a hangover prevention and/or treatment composition comprising a sodium salt and additionally comprising a phosphate salt is disclosed.

In another exemplary embodiment, a hangover prevention and/or treatment composition comprising a sodium salt and additionally comprising a glutamate salt (e.g., sodium glutamate) is disclosed.

In another exemplary embodiment, a hangover prevention and/or treatment composition comprising a sodium salt and additionally comprising a chloride salt is disclosed.

In another exemplary embodiment, a hangover prevention and/or treatment composition comprising a sodium salt and additionally comprising a carbonate salt is disclosed.

In another exemplary embodiment, a hangover prevention and/or treatment composition comprising a sodium salt and additionally comprising vitamin B12 is disclosed.

In another exemplary embodiment, a hangover prevention and/or treatment composition comprising a sodium salt and additionally comprising activated carbon is disclosed. In another exemplary embodiment, the activated carbon is at least partially soluble in water.

In another exemplary embodiment, a hangover prevention and/or treatment composition comprising a sodium salt, a sugar, and additionally comprising a citrate salt is disclosed.

In another exemplary embodiment, a hangover prevention and/or treatment composition comprising a sodium salt and additionally comprising a calcium salt is disclosed.

In another exemplary embodiment, a hangover prevention and/or treatment composition comprising a sodium salt, a carbonate salt, and a calcium salt is disclosed.

In another exemplary embodiment, a hangover prevention and/or treatment composition comprising a sodium salt and additionally comprising a potassium salt is disclosed.

In another exemplary embodiment, a hangover prevention and/or treatment composition comprising a sodium salt, a potassium salt, and additionally comprising a citrate salt is disclosed.

In another exemplary embodiment, a hangover prevention and/or treatment composition comprising a sodium salt, a potassium salt, a citrate salt, and additionally comprising a sugar is disclosed.

In another exemplary embodiment, a hangover prevention and/or treatment composition comprising a sodium salt, a potassium salt, a citrate salt, a sugar, and additionally comprising a phosphate salt is disclosed.

In another exemplary embodiment, a hangover prevention and/or treatment composition comprising a sodium salt and additionally comprising glycerol is disclosed.

Other exemplary embodiments of the invention also provide formulations for preventing and/or treating hangovers, such as, for example, formulations made from any of the above-mentioned compositions. In the description below, it should be understood that, within the context of the present invention, administering certain salt(s) in certain amounts and/or concentrations has been found to both prevent the onset of a hangover and/or treat or alleviate the symptoms of a hangover after the onset of the hangover has occurred. Accordingly, each of the methods of prevention described below may, in certain exemplary embodiments, be performed prior to the onset of a hangover in order to prevent the onset of a hangover. In other exemplary embodiments where prophylactic measures are not used and the onset of a hangover occurs, the methods described below may also be used to treat or alleviate the symptoms of a hangover after its onset.

For example, in one embodiment, the hangover prevention and/or treatment formulation comprises an aqueous solution comprising a sodium salt.

In another exemplary embodiment, the hangover prevention and/or treatment formulation comprises an aqueous solution comprising a sodium salt and a bicarbonate salt.

In another exemplary embodiment, the hangover prevention and/or treatment formulation comprises an aqueous solution comprising a sodium salt and a phosphate salt.

In another exemplary embodiment, the hangover prevention and/or treatment formulation comprises an aqueous solution comprising a sodium salt and a citrate salt.

In another exemplary embodiment, the hangover prevention and/or treatment formulation comprises an aqueous solution comprising a sodium salt and a chloride salt.

In another exemplary embodiment, the hangover prevention and/or treatment formulation comprises an aqueous solution comprising a sodium salt and a carbonate salt.

In another exemplary embodiment, the hangover prevention and/or treatment formulation comprises an aqueous solution comprising a sodium salt and vitamin B12.

In another exemplary embodiment, the hangover prevention and/or treatment formulation comprises an aqueous solution comprising a sodium salt and activated carbon, wherein the activated carbon is at least partially soluble in water.

In another exemplary embodiment, the hangover prevention and/or treatment formulation comprises an aqueous solution comprising a sodium salt and sugar.

In another exemplary embodiment, the hangover prevention and/or treatment formulation comprises an aqueous solution comprising a sodium salt and a calcium salt.

In another exemplary embodiment, the hangover prevention and/or treatment formulation comprises an aqueous solution comprising a sodium salt, a carbonate salt, and a calcium salt.

In another exemplary embodiment, the hangover prevention and/or treatment formulation comprises an aqueous solution comprising a sodium salt and a potassium salt.

In another exemplary embodiment, the hangover prevention and/or treatment formulation comprises an aqueous solution comprising a sodium salt and a potassium salt.

In another exemplary embodiment, the hangover prevention and/or treatment formulation comprises an aqueous solution comprising a sodium salt, a potassium salt, and a citrate salt.

In another exemplary embodiment, the hangover prevention and/or treatment formulation comprises an aqueous solution comprising a sodium salt, a potassium salt, a citrate salt, and a phosphate salt.

In another exemplary embodiment, the hangover prevention and/or treatment formulation comprises an aqueous solution comprising a sodium salt, a potassium salt, a citrate salt, a phosphate salt, and a sugar.

In another exemplary embodiment, the hangover prevention and/or treatment formulation comprises an aqueous solution comprising a sodium salt, a potassium salt, a citrate salt, a phosphate salt, a sugar, and vitamin B12.

In another exemplary embodiment, the hangover prevention and/or treatment formulation comprises an aqueous solution comprising a sodium salt, a potassium salt, a citrate salt, a phosphate salt, a sugar, vitamin B12, and a chloride salt. In at least one exemplary embodiment, the sugar is comprised of a mixture of sucrose and glucose.

In another exemplary embodiment, the hangover prevention and/or treatment formulation comprises an aqueous solution comprising a sodium salt and glycerol.

In one exemplary embodiment of the present invention, the electrolytes (also known to those skilled in the art as “salts”) are administered in a formulation that allows administration of more than about 200 mg of at least one electrolyte (e.g., a sodium salt) in less than about 100 mL of liquid (e.g., water).

In another exemplary embodiment of the present invention, the electrolytes are administered in a formulation that allows administering more than about 400 mg of at least one electrolyte (e.g., a sodium salt) in less than about 100 mL of liquid (e.g., water).

In another exemplary embodiment of the present invention, the electrolytes are administered in a formulation that allows administering more than about 600 mg of at least one electrolyte (e.g., a sodium salt) in less than about 100 mL of liquid (e.g., water).

In another exemplary embodiment of the present invention, the electrolytes are administered in a formulation that allows administering more than about 800 mg of at least one electrolyte (e.g., a sodium salt) in less than about 100 mL of liquid (e.g., water).

In another exemplary embodiment, any of the formulations contemplated herein additionally comprise a molecule that fluoresces when exposed to a black light. Such fluorescence may give the formulation and/or the drink containing the formulation the subjective appearance of glowing.

In one exemplary embodiment, the formulation may be provided as a concentrated salt solution, such as greater than about 0.1 M, greater than about 0.2 M, greater than about 0.4 M, greater than about 0.6 M, greater than about 0.8 M, greater than about 1 M, or greater than about 2 M, with respect to the concentration of the salt, such as sodium, in the solution.

In another exemplary embodiment, the formulation that is provided as a concentrated salt solution is provided in a unit dose form such as a single serving or “shot” sized portion. By way of example, the single serving portion ranges from about 1 to about 12 oz, such as, for example, about 7 to about 9 oz, such as about 8.4 oz. In another embodiment, the single serving portion ranges from about 5 to about 7 oz, such as, for example, about 6.5 oz.

In one exemplary embodiment, the hangover prevention and/or treatment formulation comprises an aqueous solution comprising a sodium salt, wherein said sodium salt is present in a concentration ranging from about 20 mM to about 0.4 M, from about 50 mM to about 0.3 M, or from about 100 mM to about 0.2 M.

In another exemplary embodiment, the hangover prevention and/or treatment formulation comprises an aqueous solution comprising a sodium salt, wherein said sodium salt is present in a concentration ranging from about 50 mM to about 200 mM.

In another exemplary embodiment, the hangover prevention and/or treatment formulation comprises an aqueous solution comprising a sodium salt and a bicarbonate salt, wherein said sodium salt is present in a concentration ranging from about 20 mM to about 0.4 M, from about 50 mM to about 0.3 M, or from about 100 mM to about 0.2 M. In another exemplary embodiment, the hangover prevention and/or treatment formulation comprises an aqueous solution comprising a sodium salt and a bicarbonate salt, wherein said sodium salt is present in a concentration ranging from about 50 mM to about 200 mM.

In another exemplary embodiment, the hangover prevention and/or treatment formulation comprises an aqueous solution comprising a sodium salt and a chloride salt, wherein said sodium salt is present in a concentration ranging from about 20 mM to about 0.4 M, from about 50 mM to about 0.3 M, or from about 100 mM to about 0.2 M. In another exemplary embodiment, the hangover prevention and/or treatment formulation comprises an aqueous solution comprising a sodium salt and a chloride salt, wherein said sodium salt is present in a concentration ranging from about 50 mM to about 200 mM.

In another exemplary embodiment, the hangover prevention and/or treatment formulation comprises an aqueous solution comprising a sodium salt and a glutamate salt, wherein said sodium salt is present in a concentration ranging from about 20 mM to about 0.4 M, from about 50 mM to about 0.3 M, or from about 100 mM to about 0.2 M. In another exemplary embodiment, the hangover prevention and/or treatment formulation comprises an aqueous solution comprising a sodium salt and a glutamate salt, wherein said sodium salt is present in a concentration ranging from about 50 mM to about 200 mM.

In another exemplary embodiment, the hangover prevention and/or treatment formulation comprises an aqueous solution comprising a sodium salt and a carbonate salt, wherein said sodium salt is present in a concentration ranging from about 20 mM to about 0.4 M, from about 50 mM to about 0.3 M, or from about 100 mM to about 0.2 M. In another exemplary embodiment, the hangover prevention and/or treatment formulation comprises an aqueous solution comprising a sodium salt and a carbonate salt, wherein said sodium salt is present in a concentration ranging from about 50 mM to about 200 mM.

In another exemplary embodiment, the hangover prevention and/or treatment formulation comprises an aqueous solution comprising a sodium salt and activated carbon, wherein the activated carbon is at least partially soluble in water and wherein said sodium salt is present in a concentration ranging from about 20 mM to about 0.4 M, from about 50 mM to about 0.3 M, or from about 100 mM to about 0.2 M. In another exemplary embodiment, the hangover prevention and/or treatment formulation comprises an aqueous solution comprising a sodium salt and activated carbon, wherein the activated carbon is at least partially soluble in water and wherein said sodium salt is present in a concentration ranging from about 50 mM to about 200 mM. By “at least partially in water,” it is meant that at least 1% by mass of the activated carbon dissolves in water. This may be assayed by any method known to those of skill in the art, such as, for example, stirring the activated carbon in water, then filtering the suspension through filter paper, and then evaporating the filtrate to determine if any of the carbon passed through the filter paper as an aqueous solution.

In another exemplary embodiment, the hangover prevention and/or treatment formulation comprises an aqueous solution comprising a sodium salt and a sugar, wherein said sodium salt is present in a concentration ranging from about 20 mM to about 0.4 M, from about 50 mM to about 0.3 M, or from about 100 mM to about 0.2 M. In another exemplary embodiment, the hangover prevention and/or treatment formulation comprises an aqueous solution comprising a sodium salt and a sugar, wherein said sodium salt is present in a concentration ranging from about 50 mM to about 200 mM.

In another embodiment, the hangover prevention and/or treatment formulation comprises an aqueous solution comprising a sodium salt and a calcium salt, wherein said sodium salt is present in a concentration ranging from about 20 mM to about 0.4 M, from about 50 mM to about 0.3 M, or from about 100 mM to about 0.2 M. In another exemplary embodiment, the hangover prevention and/or treatment formulation comprises an aqueous solution comprising a sodium salt and a calcium salt, wherein said sodium salt is present in a concentration ranging from about 50 mM to about 200 mM.

In another exemplary embodiment, the hangover prevention and/or treatment formulation comprises an aqueous solution comprising a sodium salt, a carbonate salt, and a calcium salt, wherein said sodium salt is present in a concentration ranging from about 20 mM to about 0.4 M, from about 50 mM to about 0.3 M, or from about 100 mM to about 0.2 M. In another exemplary embodiment, the hangover prevention and/or treatment formulation comprises an aqueous solution comprising a sodium salt, a carbonate salt, and a calcium salt, wherein said sodium salt is present in a concentration ranging from about 50 mM to about 200 mM.

In another exemplary embodiment, the hangover prevention and/or treatment formulation comprises an aqueous solution comprising a sodium salt and a potassium salt, wherein said sodium salt is present in a concentration ranging from about 20 mM to about 0.4 M, from about 50 mM to about 0.3 M, or from about 100 mM to about 0.2 M. In another exemplary embodiment, the hangover prevention and/or treatment formulation comprises an aqueous solution comprising a sodium salt and a potassium salt, wherein said sodium salt is present in a concentration ranging from about 50 mM to about 200 mM.

In another exemplary embodiment, the hangover prevention and/or treatment formulation comprises an aqueous solution comprising a sodium salt, a potassium salt, and a chloride salt, wherein said sodium salt is present in a concentration ranging from about 20 mM to about 0.4 M, from about 50 mM to about 0.3 M, or from about 100 mM to about 0.2 M. In another exemplary embodiment, the hangover prevention and/or treatment formulation comprises an aqueous solution comprising a sodium salt, a potassium salt, and a chloride salt, wherein said sodium salt is present in a concentration ranging from about 50 mM to about 200 mM.

In another exemplary embodiment, the hangover prevention and/or treatment formulation comprises an aqueous solution comprising a sodium salt, a citrate salt, and a potassium salt, wherein said sodium salt is present in a concentration ranging from about 20 mM to about 0.4 M, from about 50 mM to about 0.3 M, or from about 100 mM to about 0.2 M. In another exemplary embodiment, the hangover prevention and/or treatment formulation comprises an aqueous solution comprising a sodium salt, a citrate salt, and a potassium salt, wherein said sodium salt is present in a concentration ranging from about 50 mM to about 200 mM.

In another exemplary embodiment, the hangover prevention and/or treatment formulation comprises an aqueous solution comprising a sodium salt, a citrate salt, a potassium salt, and a phosphate salt, wherein said sodium salt is present in a concentration ranging from about 20 mM to about 0.4 M, from about 50 mM to about 0.3 M, or from about 100 mM to about 0.2 M. In another exemplary embodiment, the hangover prevention and/or treatment formulation comprises an aqueous solution comprising a sodium salt, a citrate salt, a potassium salt, and a phosphate salt wherein said sodium salt is present in a concentration ranging from about 50 mM to about 200 mM.

In another exemplary embodiment, the hangover prevention and/or treatment formulation comprises an aqueous solution comprising a sodium salt, a citrate salt, a potassium salt, a phosphate salt, and a sugar, wherein said sodium salt is present in a concentration ranging from about 20 mM to about 0.4 M, from about 50 mM to about 0.3 M, or from about 100 mM to about 0.2 M. In another exemplary embodiment, the hangover prevention and/or treatment formulation comprises an aqueous solution comprising a sodium salt, a citrate salt, a potassium salt, a phosphate salt, and a sugar, wherein said sodium salt is present in a concentration ranging from about 50 mM to about 200 mM.

In another exemplary embodiment, the hangover prevention and/or treatment formulation comprises an aqueous solution comprising a sodium salt, a potassium salt, and a chloride salt, wherein said sodium salt is present in a concentration ranging from about 8 mM to about 8 M, from about 8 mM to about 1 M, from about 16 mM to about 0.6 M, from about 20 mM to about 0.4 M, from about 50 mM to about 0.3 M, or from about 100 mM to about 0.2 M, and further wherein the ratio of the sodium salt to the potassium salt ranges from about 10 to 1, such as from about 20 to 1, or from about 40 to 1.

In another exemplary embodiment, the hangover prevention and/or treatment formulation comprises an aqueous solution comprising a sodium salt and glycerol, wherein said sodium salt is present in a concentration ranging from about 20 mM to about 0.4 M, from about 50 mM to about 0.3 M, or from about 100 mM to about 0.2 M. In another exemplary embodiment, the hangover prevention and/or treatment formulation comprises an aqueous solution comprising a sodium salt and glycerol, wherein said sodium salt is present in a concentration ranging from about 50 mM to about 200 mM.

In another exemplary embodiment, the formulation of the present invention is a liquid concentrate. Such a liquid concentrate may, for example, be used as a mixer in a cocktail. The term “mixer,” within the context of the present invention, refers to a nonalcoholic beverage, such as sour mix, simple syrup, mojito mix, daiquiri mix, margarita mix, etc., which is conventionally used as a component or additive in cocktails. Exemplary, non-limiting examples of mixers can be found in bartending manuals. See, e.g., Cunningham, S. K., The Bartender's Black Book, Eighth Edition. In one exemplary embodiment of the invention, the liquid concentrate or mixers of the present invention contain a sodium salt concentration ranging from about 20 mM to about 0.4 M, from about 50 mM to about 0.3 M, from about 100 mM to about 0.5 M, or from about 200 mM to about 1 M. In another exemplary embodiment, the liquid concentrate or mixers of the invention comprise an aqueous solution comprising a sodium salt, wherein said sodium salt is present in a concentration ranging from about 50 mM to about 200 mM.

In one exemplary embodiment, a formulation of the present invention is a carbonated beverage. For example, the carbonated beverage may comprise a sodium salt in a concentration ranging from about 40 mM to about 0.2 M. In another exemplary embodiment, the carbonated beverage comprises a sodium salt in a concentration ranging from about 60 mM to about 0.15 M. In another exemplary embodiment, the carbonated beverage comprises a sodium salt in a concentration ranging from about 0.1-0.2 M. In another exemplary embodiment, the carbonated beverage comprises a sodium salt concentration of not less than 60 mM.

This invention also provides methods for preparing the formulations of the invention as described above.

In one exemplary embodiment, methods of preparing formulations of the present invention comprise adding, for example to an aqueous medium such as a beverage comprising water and/or carbonated water, a concentrated aqueous solution comprising one or more salts as described herein and/or one or more sugars as described herein, and optionally other additives such as flavoring agents.

In another exemplary embodiment, methods of preparing formulations of the present invention comprise adding, for example to an aqueous medium such as a beverage comprising water and/or carbonated water, a composition of the invention, such as, for example, as a concentrated liquid or tablet.

This invention provides methods of preventing and/or treating a hangover, for example the manifestations of headache and/or nausea. For example, the invention described herein provides methods of preventing and/or treating a hangover comprising administering one or more of the compositions and/or formulations as described herein to a person in need thereof, such as, for example, as a person with a BAC of greater than about 0.01%.

In another exemplary embodiment, a method of preventing and/or treating a hangover comprises administering one or more of the compositions and/or formulations as described herein to a person with a BAC of greater than about 0.1%.

In another exemplary embodiment, a method of preventing and/or treating a hangover comprises administering one or more compositions and/or formulations as described herein to a person with a BAC of greater than about 0.01%, wherein the one or more of the compositions and/or formulations is/are administered multiple times surrounding the period of time when the person is consuming alcoholic beverages, such as, for example, one or more times during any of the times prior to, during, and after the person is consuming alcoholic beverages.

In various exemplary embodiments disclosed herein, methods of preventing and/or treating a hangover comprise administering, to the person consuming alcoholic beverages, one or more of the above compositions and/or formulations as described herein one or more times during the period of time when the person's BAC is greater than about 0.01%. In another exemplary embodiment, a method comprises administering a composition as described herein by delivering the composition in tablet form to an aqueous medium in order to prepare a liquid formulation, and thereafter administering the liquid formulation to a person in need thereof.

In another exemplary embodiment, a method of preventing and/or treating a hangover comprises determining the approximate amount of alcohol consumed by a person, using this approximation to determine the amount of composition and/or formulation as described herein to be administered, and administering an amount of a composition or a formulation based on the approximate amount of alcohol consumed.

In another exemplary embodiment, a method of preventing and/or treating a hangover comprises determining the approximate quantity of electrolytes lost by a person during a period of consuming alcohol (e.g., by determining the amount of urine excreted by a person during a period of consuming alcohol), using this approximation to determine the amount of composition and/or formulation as described herein to be administered, and administering an amount of a composition or a formulation based on the approximate quantity of electrolytes lost. Measuring the concentration of electrolytes in urine is easily accomplished by those of skill in the art using routine procedures, for example by quantitative ion-exchange methods known since at least 1954. See Vanatta, J. C. et al, Journal of Biological Chemistry, 1954, 212: 599.

In another exemplary embodiment, the method of preventing and/or treating a hangover comprises administering a concentrated composition and/or formulation (e.g., powder or gel) as described herein to a beverage in order to increase the electrolyte concentration thereof. In another exemplary embodiment, the beverage to which the composition and/or formulation as described herein is added comprises ethanol. In another exemplary embodiment, the beverage to which the composition and/or formulation as described herein is added is beer.

The present invention also provides for dry varieties of the above-described formulations. Exemplary formulations may include, for example, powders, tablets, pills, etc.

In one exemplary embodiment, the dry form (e.g., tablet or powder) comprising one or more of the compositions of the invention effervesces when it is added to water.

In another exemplary embodiment, one or more of the compositions and/or formulations of the invention are provided in a unit dose form, wherein said unit dose form comprises a concentrated powder or gel, and which may, in an exemplary embodiment, be administered directly by mouth or first added to a beverage. In another exemplary embodiment, the formulation is an extended-release or time-released tablet or pill, which may be administered alone or in combination with other compositions and/or formulations of the invention. Within the context of the present invention, “extended release” or “time-released” are intended to embody formulations that are designed to release salt into the subject (i.e., person consuming alcoholic beverages) over an extended period of time, such as, for example, a period of greater than about one hour. For example, extended release formulations, controlled release formulations, and the like, may be those which release, e.g., sodium chloride, sodium carbonate, sodium phosphate, sodium citrate, sodium bicarbonate, etc., into the subject's circulating fluid volume.

In one exemplary embodiment, the method of treating and/or preventing a hangover comprises administering one or more time-released formulations of this invention prior to, during, or after the person's BAC reaches about 0.01%.

In another exemplary embodiment, the invention provides for a kit comprising a composition and/or formulation of the invention in one or more unit dose forms. In one exemplary embodiment, the kit may comprise one or more unit dose forms in a single serving drink formulation. In another exemplary embodiment, the kit may comprise one or more unit dose forms in a dry formulation, such as a powder or pill, for example. In another exemplary embodiment, the kit may comprise one or more tablets, wherein each tablet comprises (1) about 350 mg to about 1.5 g of sodium, (2) NaHCO₃ and/or KHCO₃, and (3) an acid suitable for human consumption, such as, for example, citric acid or ascorbic acid. In another exemplary embodiment, the kit may package together one or more tablets in a cylinder or tube with the ingredients and directions for use therein.

It is noted that, as used in this specification and the appended claims, the singular forms “a,” “an,” and “the,” are intended to include plural referents unless expressly and unequivocally limited to one referent, and vice versa. Thus, by way of example only, reference to “a salt” can refer to one or more salts. As used herein, the term “include” and its grammatical variants are intended to be non-limiting, such that recitation of items in a list is not to the exclusion of other like items that can be substituted or added to the listed items.

It will be apparent to those skilled in the art that various modifications and variation can be made to the programs and methods of the present disclosure without departing from the scope its teachings. Other embodiments of the disclosure will be apparent to those skilled in the art from consideration of the specification and practice of the teachings disclosed herein. It is intended that the embodiments described in the specification be considered as exemplary only.

### EXAMPLES

The following examples are not intended to be limiting but rather represent general methods through which the formulations of this invention may be made. It should be appreciated that the other salts of this invention may be substituted by using the guidance pertaining to concentration and/or combination provided herein. For example, the examples below refer to sodium chloride as the exemplified sodium salt. However, an alternative sodium salt, such as sodium citrate may be used by using the guidance pertaining to concentration and/or combination provided in the specification. Additionally, one of ordinary in the art will recognize that various other solution concentrations (e.g., sodium concentration, phosphate concentration, sugar concentration, citrate concentration, potassium concentration) may be substituted by using the guidance provided herein and generally accepted relationships pertaining to formula weights, solution volumes, and molar concentrations.
1. To a 2 L flask was added 1.1 g of NaCl and 1 L of water. The resulting solution was stirred until all of the contents dissolved completely.
2. To the solution in 1, was added 300 mg of KPO₄H₂. The resulting solution was stirred until all of the contents dissolved.
3. To the solution in 2 was added 120 g of sucrose. The resulting solution was stirred until the contents fully dissolved.
4. To the solution in 1 was added 100 mg of KCl. The resulting solution was stirred until all of the contents dissolved.
5. To the solution in 1 was added 30 mL of glycerol. The glycerol formed an oil at the bottom of the flask, which disappeared upon stirring.
6. To the solution in 1 was added KHCO₃. The resulting solution was stirred until all of the contents dissolved.
7. To the solution in 1 was added CaCO₃. The resulting solution was stirred until all of the contents dissolved.
8. To the solution in 1 was added activated carbon, which resulted in a suspension.
9. To a 2 L flask was added 2.2 g of NaCl, 0.9 g of KPO₄H₂, and 1 L of water. The resulting mixture was stirred until all of the contents dissolved completely.
10. To the solution in 9 was added 120 g of sucrose. The resulting mixture was dissolved until all of the contents dissolved completely.
11. 200 mg of NaCl and 100 mg of KCl were dry-blended together with 100 mg of NaHCO₃.
12. To a 1 L flask was added 50 g of sodium glutatmate and 1 L of water. The resulting mixture was stirred until the contents dissolved completely.
13. To the solution in 12 was added 300 mg of NaCl. The resulting mixture was stirred until the contents dissolved completely.
14. To the solution in 12 was added 500 mg of KCl. The resulting mixture was stirred until the contents dissolved completely.
15. To the solution in 12 was added 2 g of NaHCO₃. The resulting mixture was stirred until the contents dissolved completely.
16. To the solution in 12 was added 2.6 g of KHCO₃. The resulting mixture was stirred until the contents dissolved completely.
17. To a 2 L flask was added 10.0 g of NaCl and 1.0 L of water. The resulting solution was stirred until all of the contents dissolved completely.
18. To the solution in 17, was added 300 mg of KPO₄H₂. The resulting solution was stirred until all of the contents dissolved.
19. To the solution in 18 was added 120 g of sucrose. The resulting solution was stirred until the contents fully dissolved.
20. To the solution in 17 was added 100 mg of KCl. The resulting solution was stirred until all of the contents dissolved.
21. To the solution in 17 was added 30 mL of glycerol. The glycerol formed an oil at the bottom of the flask, which disappeared upon stirring.
22. To the solution in 17 was added KHCO₃. The resulting solution was stirred until all of the contents dissolved.
23. To the solution in 17 was added CaCO₃. The resulting solution was stirred until all of the contents dissolved.
24. To the solution in 17 was added activated carbon, which resulted in a suspension.
25. To a 2 L flask was added 20.1 g of NaCl, 0.9 g of KPO₄H₂, and 1 L of water. The resulting mixture was stirred until all of the contents dissolved completely.
26. To the solution in 25 was added 120 g of sucrose. The resulting mixture was dissolved until all of the contents dissolved completely.

The following exemplary studies are not intended to be limiting. Rather these examples are intended to reflect exemplary embodiments of the methods of preventing and/or treating hangovers described herein, using the exemplary formulations described above. It should be appreciated that other formulations, either expressly exemplified above or described in the specification may be substituted, following procedures similar to the procedures below. For example, in lieu of sodium chloride, an alternative sodium salt (such as, e.g., sodium citrate) may be used provided that the concentration of sodium ion adheres to the guidelines set forth in the specification. Additionally, the amount of alcohol consumed in the test group below is not intended to be limiting. Rather, these examples, using heavy drinking, were selected to demonstrate the efficacy of the formulations of this invention.
A. A group of 12 human subjects was allowed free access to light beer in a late night party environment. The subjects were all observed to participate in social “drinking games” and were estimated to consume between 12-18 12 oz beers per person throughout a period of 6 hours. Based on external behavioral manifestations, it was estimated that the BAC of each member in the group exceeded 0.1%, which comports with the number of alcoholic beverages consumed. Following the 6-hour period of drinking, each subject consumed 500 mL of the solution described above in 10. After consuming the solution, each subject was allowed to sleep for approximately 6 hours, from 2:30 am to 8:30 am, and was thereafter asked to report on the relative subjective severity of their hangover symptoms (compared to a similar period of consumption without administering the formulation), particularly headache and nausea. Of the 12 subjects, 10 reported significant improvements in hangover symptoms, compared to their prior hangover experiences after consuming commensurate quantities of alcohol. Two subjects reported neither an improvement nor a worsening of hangover symptoms. Of the 10 subjects to report improvements, 8 reported a “complete absence” of headache and/or nausea. The fatigue was assigned, at least in part, to the shortened night of sleep and/or disturbances in sleep pattern caused by the well-known affects of ethanol on REM sleep.
B. A group of 8 human subjects, including the two subjects who reported no improvement in hangover symptoms, was exposed to nearly identical drinking conditions. Each subject was administered 100 mL of formulation 10 (above) four or five times throughout the course of drinking alcoholic beverages. Each subject was allowed 6 hours of sleep. The following morning, upon waking from the 6 hours of sleep, all eight subjects reported a complete absence of headache and/or nausea. The subjects in the study all reported mild fatigue and a general feeling of decreased acumen, neither of which were investigated further. The fatigue was assigned, at least in part, to the shortened night of sleep and/or disturbances in sleep pattern caused by the well known affects of ethanol on REM sleep.
C. A group of 5 human subjects was allowed free access to regular (i.e., not light) beer for six hours (“the experimental period”). Each of the 5 human subjects was observed to consume between 10 to 12 pints of regular beer during this period of time. Each subject was observed to exhibit physical manifestations that correlated with a BAC in excess of 0.1%. Throughout the experimental period, each subject was administered the formulation 11 (above). The formulation 11 was administered to each subject after each subject had consumed his third, sixth, ninth, and (where applicable) 12th beer. The formulation 11 was either dropped into a 2-4 oz portion of beer or, alternatively, into a 2-4 oz portion of water. For each subject, the formulation 11 was allowed to dissolve completely and the resulting solution was consumed. Additionally, one hour following ceasing drinking, to each subject was administered one additional formulation of 11, above. Each subject was allowed to sleep for approximately 7 hours, from 1:30 am to 8:30 am, and was thereafter asked to report on the relative subjective severity of their hangover symptoms (compared to a similar period of consumption without administering the formulation), particularly headache and nausea. Each of the five subjects reported the absence of a “hangover.” Specifically each subject, while reporting mild fatigue, was free from both nausea and headache.
D. Two volunteers (“subjects”) were selected based on survey results which identified them as particularly susceptible to headaches and nausea beginning 4-6 hours following ceasing a period of drinking 8-12 alcoholic beverages. Each subject was allowed free access to light beer for two consecutive evenings (first and second evenings respectively) in a festive party atmosphere. On the first evening, each subject consumed 14 12 oz beers (beers 1-14 respectively) over a 6 hour period. The consumption of beverages was not evenly spaced throughout this interval and no attempt was made to quantify the frequency during particular intervals. After each of beers number 2, 4, 6, 8, 10, and 12, each subject was administered 4 oz of an aqueous solution comprising 300 mg of sodium, 30 mg of potassium, and 20 g of a mixture of sucrose and dextrose. On the morning following the first evening, each subject was asked to confirm the dosing regime outlined above and also report on the subjective intensity of their hangover symptoms. Each subject reported having no hangover, indicated by responses of “I feel fine” and “no problem at all” for the two subjects respectively. On the second evening, one subject (Subject A) consumed 18 12 oz beers (beers 1-18 respectively) over a 7 hour period. After each of beers number 2, 4, 6, 8, 10, and 12, 14, 16, and 18, Subject A was administered 4 oz of an aqueous solution comprising 300 mg of sodium, 30 mg of potassium, and 20 g of a mixture of sucrose and dextrose. Subject A vomited approximately 1 L of fluid volume following rapidly consuming beers number 12, 13, and 14 in a 10 minute period. The administration of the 4 oz solution following beer number 12 (assumed to be expelled within the vomited material) was not repeated. The above-mentioned dosage regimen was followed following the remaining beers, numbered 14, 16, and 18. The second subject (Subject B) consumed 12 12 oz beers (beers 1-12 respectively) over a 4 hour period. To Subject B was administered 4 oz of an aqueous solution comprising 300 mg of sodium, 30 mg of potassium, and 20 g of a mixture of sucrose and dextrose following each of beers 2, 4, 6, 8, 10. Subject B consumed 2 4 oz doses of an aqueous solution comprising 300 mg of sodium, 30 mg of potassium, and 20 g of a mixture of sucrose and dextrose following beer number 12. On the morning following the second evening, each subject was asked to confirm the dosing regime outlined above and also report on the subjective intensity of their hangover symptoms. On the morning following the second evening, following six hours of sleep, each subject reported having little or no hangover. Subject A reported feeling tired but free from nausea and headache. Subject B reported feeling “completely fine”.

### Exemplary Formulation

An exemplary 8.4 oz beverage formulation was prepared comprising the following:

| | | PER 8.4 OZ | |
|---|---|---|---|
| COMPONENT | PERCENT BY WEIGHT | SERVING | |
| Sucrose | 9 | 22.35 | grams |
| Glucose | 1 | 2.48 | grams |
| Sodium citrate | 0.95 | 2.35 | grams |
| Sodium chloride | 0.495 | 1.22 | grams |
| Citric acid | 0.42 | 1.04 | grams |
| Dipotassium phosphate | 0.026 | 0.06 | grams |
| Sodium benzoate | 0.0225 | 0.055 | grams |
| Gum Arabic | 0.02 | 0.049 | grams |
| Ester gum | 0.003 | 0.007 | grams |
| Natural flavors | 0.1 | 0.248 | grams |
| Yellow #5 | 0.003 | 0.007 | grams |
| Vitamin B12 | 0.0024 | 0.0059 | grams |
| Carbonated water | 87.9581 | 218.5 | oz |

1. A composition for treating and/or preventing a hangover comprising a sodium salt, wherein the sodium salt is present in the composition in an amount effective to prevent a hangover, treat a hangover, or both.

2. The composition of claim 1, additionally comprising a sugar.

3. The composition of claim 1, additionally comprising at least one of a citrate salt, a phosphate salt, a chloride salt, a potassium salt, and vitamin B12.

4. The composition of claim 2, additionally comprising at least one of a citrate salt, a phosphate salt, a potassium salt, and vitamin B12.

5. A formulation for treating and/or preventing a hangover comprising an aqueous solution comprising a composition according to claim 1.

6. The formulation for treating and/or preventing a hangover of claim 5, wherein the concentration of the sodium salt in the aqueous solution is from about 0.05-0.5 M.

7. (canceled)

8. (canceled)

9. The formulation for treating and/or preventing a hangover of claim 5, wherein the concentration of the sodium salt in the aqueous solution is greater than 0.08 M.

10. (canceled)

11. (canceled)

12. A method of treating and/or preventing a hangover comprising administering a composition according to claim 1 to a person with a blood alcohol level of greater than 0.01.

13. A method of treating and/or preventing a hangover comprising administering a formulation according to claim 5 to a person with a blood alcohol level of greater than 0.01.

14. A method of treating and/or preventing a hangover comprising administering a formulation according to claim 6 to a person with a blood alcohol level of greater than 0.01.

15. (canceled)

16. (canceled)

17. A method of treating and/or preventing a hangover comprising administering a formulation according to claim 9 to a person with a blood alcohol level of greater than 0.01.

18. (canceled)

19. (canceled)

20. (canceled)

21. (canceled)

22. (canceled)

23. (canceled)

24. (canceled)

25. (canceled)

26. (canceled)

27. (canceled)

28. A method of treating and/or preventing a hangover comprising determining the approximate amount of alcohol consumed by a person and administering an amount of a composition according to claim 1, based on the approximate amount of alcohol consumed.

29. A method of treating and/or preventing a hangover comprising determining the approximate amount of urine excreted by a person consuming alcohol and administering an amount of a composition according to claim 1, based on the approximate amount of electrolytes lost.

30. A method of treating and/or preventing a hangover comprising determining the approximate amount of alcohol consumed by a person and administering an amount of a formulation according to claim 5 based on the approximate amount of alcohol consumed.

31. A method of treating and/or preventing a hangover comprising determining the approximate amount of urine excreted by a person consuming alcohol and administering an amount of a formulation according to claim 5 based on the approximate amount of electrolytes lost.

32. A kit comprising a composition according to claim 1 in one or more unit dose forms.

33. A kit comprising a formulation according to claim 5 in one or more unit dose forms.

34. The kit of claim 33, wherein the unit dose form is a single serving drink formulation.

35. The kit of claim 34, wherein the single serving drink formulation is from about 50 mL to about 200 mL in volume.

36. The formulation according to claim 5, additionally comprising at least one of a molecule that fluoresces when exposed to a black light, a flavoring agent, or a preserving agent.
